(19) United States
(12) Patent Application Publication
(10) Pub. No.: US 2025/0381138 A1
(43) Pub. Date: Dec. 18, 2025

(54) Non-opioid analgesic formulations of nanoemulsions

(71) Applicant: Duquesne University of the Holy Spirit, PITTSBURGH, PA (US)

(72) Inventor: Jelena M. Janjic, Pittsburgh, PA (US)

(73) Assignee: Duquesne University of the Holy Spirit, PITTSBURGH, PA (US)

(21) Appl. No.: 19/213,185

(22) Filed: May 20, 2025

Related U.S. Application Data
(60) Provisional application No. 63/649,729, filed on May 20, 2024.

(51) Int. Cl. A61K 9/107; A61K 9/19; A61K 31/137; A61K 31/167; A61K 31/197; A61K 31/433; A61K 31/4402; A61K 31/46; A61K 31/5513; A61K 31/635; A61P 29/00

(52) U.S. Cl. CPC A61K 9/1075; A61K 9/19; A61K 31/137; A61K 31/167; A61K 31/197; A61K 31/433; A61K 31/4402; A61K 31/46; A61K 31/5513; A61K 31/635; A61P 29/00

(57) ABSTRACT

The present nanoemulsions are a delivery system for virtually any non-opioid active agent for pain, and contain: a hydrocarbon lipid; a perfluorocarbon; water and/or buffered saline; a nonionic surfactant; and optionally a quaternary ammonium compound and/or an additional lipid. Droplets of the non-opioid analgesic formulations have a diameter of 150 nm or less, and preferably range from 90 nm to about 120 nm and have shelf stability for at least twelve months. The nanoemulsions are suitable for a wide variety of routes of administration, including but not limited to IV and parenteral, the latter having particular battlefield and war zone suitability.

### CROSS-REFERENCE TO RELATED APPLICATION

This application claims priority to U.S. Provisional Patent Application No. 63/649,729, filed May 20, 2024, which is entirely incorporated here.

### STATEMENT OF FEDERALLY SPONSORED RESEARCH

This invention was made with government support under W81XWH-19-1-0828 awarded by the Congressionally Directed Medical Research Programs. This invention was made with government support under W81XWH-20-1-0276 awarded by the Congressionally Directed Medical Research Programs. This invention was made with government support under CDMRP W81XWH-20-1-0730 awarded by the Congressionally Directed Medical Research Programs. This invention was made with government support under W81XWH-20-2-0024 awarded by the Congressionally Directed Medical Research Programs. This invention was made with government support under CDMRP W81XWH-20-1-0854 awarded by the Congressionally Directed Medical Research Programs. This invention was made with government support under CDMRP W81XWH-20-1-0769 awarded by the Congressionally Directed Medical Research Programs. This invention was made with government support under CDMRP W81XWH-21-1-0197 awarded by the Congressionally Directed Medical Research Programs. This invention was made with government support under FA8650-20-C-6215 awarded by the U.S. Air Force Medical Support Agency. This invention was made with government support under FA8650-20-C-6224 awarded by the U.S. Air Force Medical Support Agency. The government has certain rights in the invention.

### BACKGROUND OF THE INVENTION

A large majority of casualties sustained during combat result from blast injuries with more than half affecting extremities, and virtually all resulting in peripheral nerve injuries. Most war fighters experiencing polytrauma sustain nerve injuries and such neuromuscular polytrauma may result in sensorimotor deficits and often intractable pain, dramatically reducing quality of life. Additionally, pain such as chronic pain is the leading cause of disability and reduced readiness in military-related personnel and opioid use is often relied on for pain management resulting in an “epidemic” of prescription opioid abuse among veterans. However, non-opioid alternatives (e.g., NSAIDs, tricyclics, gabapentin, buprenorphine) are often found to be suboptimal due to varied efficacies and systemic, performance-limiting side effects. Accordingly, a need remains for a delivery system for non-opioid active agents which is virtually instantaneous, particularly well suited for use in emergencies in the field, and for which the side effects of oral pain medications is minimized or eliminated.

### SUMMARY OF THE INVENTION

In order to meet this need, the present invention is a nanoemulsion delivery system for any known non-opioid active agent for pain, muscular relaxation, nerve calming or any other drug known to have utility in providing patient comfort after injury. The specifics of the nanoemulsion delivery system set it apart from generic nanoemulsions, in that the present nanoemulsion has both a specific chemical make-up and important particle dimensions. The present nanoemulsion will always contain: a hydrocarbon lipid; a perfluorocarbon; water and/or buffered saline; a nonionic surfactant; and optionally a quaternary ammonium compound and/or an additional lipid. Droplets of the non-opioid analgesic formulations have a diameter of 150 nm or less, and preferably range from 90 nm to about 120 nm and have shelf stability even under extreme temperature conditions for at least twelve months. By “extreme temperature” is meant anywhere from 40 degrees F. up to about 120 degrees F., with excursions permitted above and below these range limits. The importance of the shelf stability under challenging conditions is the utility of these compositions in the field, including combat locations, where controlled environmental temperatures are virtually unattainable. As a metric of stability, the present droplets do not vary from their initial diameters by more than 20% over a one-year's storage period. The stable nanoemulsions of the present invention are suitable for virtually any route of administration, but one non-limiting advantageous treatment mode is parenteral administration, generally in the area of or adjacent traumatic injury. The present stable composition when administered parenterally near or at the injury site do not merely avoid the hepatic first-pass effect, they provide rapid and localized absorption right in the area where pain relief is needed most urgently.

### BRIEF DESCRIPTION OF THE DRAWINGS

FIG. 1a and FIG. 1b show macrophage infiltration-macrophages are a key cellular target in neuro-muscular trauma—in contused muscle associated with hypersensitivity severity revealed by NIRF signal;

FIG. 1c and FIG. 1d show macrophages surrounding injured muscle fibers;

FIG. 1e shows that sustained pain relief (greater than 40 days) was achieved in this model following a single dose of CXB-NE;

FIG. 2a and FIG. 2b show that locally administered CXB-NE promoted axon survival and myelin preservation following SN-TR injury while untreated controls were associated with extensive myelin disruption and axonal loss;

FIG. 3a illustrates that the NIRF signal was quantified as a surrogate measure of neuromuscular inflammation; and

FIG. 3b illustrates that increased Peak Amplitude (PA) is associated with axonal sprouting, which is evidence of regenerating axons.

### DETAILED DESCRIPTION OF THE INVENTION

As described above, the gravamen of the present invention inheres in the specifics of the nanoemulsion delivery system, which set it apart from generic nanoemulsions. The present nanoemulsion has both a specific chemical make-up and important particle dimensions. The present nanoemulsion will always contain: a hydrocarbon lipid; a perfluorocarbon; water and/or buffered saline; a nonionic surfactant; and optionally a quaternary ammonium compound and/or an additional lipid. Droplets of the non-opioid analgesic formulations have a diameter of 150 nm or less, and preferably range from 90 nm to about 120 nm and have shelf stability even under extreme temperature conditions for at least twelve months. By “extreme temperature” is meant anywhere from 40 degrees F. up to about 120 degrees F., with excursions permitted above and below these range limits. The importance of the shelf stability under challenging conditions is the utility of these compositions in the field, including combat locations, where environmental conditions often cannot be controlled. As a metric of stability, the present droplets do not vary from their initial diameters by more than 20% over a one-year's storage period. The stable nanoemulsions of the present invention are suitable for virtually any route of administration, but one non-limiting advantageous treatment mode is parenteral administration, generally in the area of or adjacent traumatic injury. The present stable composition when administered parenterally near or at the injury site do not merely avoid the hepatic first-pass effect, they provide rapid and localized absorption right in the area where pain relief is needed most urgently. Virtually any active agent in the pain relief or related indication areas (muscle relaxers such as gabapentin, tizanidine, etc.) may be delivered with the present nanoemulsions, to new and surprisingly improved effect.

In the present invention, stabilized nanoemulsions may be formulated for parenteral administration for effective pain management, with particular applications in battlefield uses, such during acute stages, during military treatment facility care, and during rehabilitation and recovery. Stabilized nanoemulsions may be formulated for parenteral administration as single-dose, long-acting analgesic without cardiovascular/respiratory effects, without performance-limiting effects (e.g. sedation) or interference with other treatments and interventions, that can be quickly administered on the battlefield by a medic to provide effective pain control. In particular, the stabilized nanoemulsions may be formulated as non-opioid nanomedicine analgesic (without any opioid) as single dose, parenteral administration (i.v. and/or s.c.), to provide effective pain relief without systemic adverse effects.

The non-opioid analgesic formulations of the present invention may achieve injury-specific, targeted immunomodulation, while producing analgesia, and promoting neuromuscular regeneration. In particular, the non-opioid analgesic formulations may achieve effective trauma pain relief lasting at least 72 hours following a single parenteral dose for battlefield trauma devoid of systemic and performance-limiting side effects or cardiovascular and respiratory liabilities.

Stabilized nanoemulsions, such as perfluorocarbon, hydrocarbon/lipid or perfluorocarbon/hydrocarbon or perfluorocarbon/lipid complex nanoemulsions, may be formulated in sterile forms, as liquid and powdered forms, and produced with Generally Recognized as Safe (GRAS) ingredients. The resulting non-opioid analgesic formulations may be packaged for the battlefield, resource-limited, and austere environments to treat trauma pain as either liquids or lyophilized stabilized powders in sterile form.

The non-opioid analgesic formulations of the present invention may be prepared via scalable high shear emulsification processes (e.g. sonication, microfluidization) in stabilized liquid form, which can be further lyophilized into a stable, sterile, powdered form. The non-opioid analgesic formulations in both liquid and powdered forms may be used as a single dose at the time of injury and achieve effective analgesia without systemic and/or performance-limiting side effects and promotes wound healing through immunomodulation. The non-opioid analgesic formulations may be administered i.v. or locally. The non-opioid analgesic formulations may provide ongoing pain-relief, accelerated wound healing, and reduced inflammation following a single dose administration. Additionally, the non-opioid analgesic formulations may achieve macrophage-specific COX-2 inhibition at a very low dose (e.g., <2 mg/kg).

The non-opioid analgesic formulations may include stabilized perfluorocarbon nanoemulsions, hydrocarbon/lipid nanoemulsions, perfluorocarbon/hydrocarbon nanoemulsions and may be prepared by incorporating a non-opioid active analgesic ingredient into the nanoemulsion, an excipient and/or one or more other accessory ingredients, and then, if desirable or necessary, dividing, shaping, and/or packaging the product into a desired single- or multi-dose unit.

The non-opioid analgesic formulations may be prepared, packaged, and/or sold in bulk, as a single unit dose, and/or as a plurality of single unit doses. As used herein, a “unit dose” is a discrete amount of the pharmaceutical composition comprising a predetermined amount of the active ingredient (e.g., lipid nanoparticle). The amount of the active ingredient is generally equal to the dosage of the active ingredient which would be administered to a subject and/or a convenient fraction of such a dosage, for example, one-half or one-third of such a dosage.

The non-opioid analgesic formulations may be prepared in a variety of forms suitable for a variety of routes and methods of administration. For example, formulations may be prepared in liquid dosage forms (e.g., emulsions, microemulsions, nanoemulsions, solutions, suspensions, syrups, and elixirs), injectable forms, solid dosage forms (e.g., powders, and granules), dosage forms for topical and/or transdermal administration (e.g., ointments, pastes, creams, lotions, gels, powders, solutions, sprays, inhalants, and patches), suspensions, powders, and other forms.

Liquid dosage forms for parenteral administration include, but are not limited to, pharmaceutically acceptable emulsions, microemulsions, nanoemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include additional therapeutics and/or prophylactics, additional agents such as wetting agents, emulsifying and suspending agents, sweetening, flavoring, and/or perfuming agents.

In parenteral administration, compositions may be mixed with solubilizing agents such as Cremophor®, alcohols, oils, modified oils, glycols, polysorbates, cyclodextrins, polymers, and/or combinations thereof.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables.

Injectable formulations may be sterilized, for example, by filtration through a bacterial-retaining filter, and/or by incorporating sterilizing agents in the form of sterile solid compositions which can be dissolved or dispersed in sterile water or other sterile injectable medium prior to use.

The non-opioid analgesic nanoemulsion formulations were developed to provide the following characteristics: 1) colloidal and chemical stability related to shelf-life and applicability in challenging environments (e.g. exposure to changing temperatures, high or low temperatures during storage and use, shipment); 2) nanoemulsion components including perfluorocarbons and surfactants minimize engagement of the immune system; 3) manufacturing process established for production on large-scale and batch to batch quality control throughout the product life-time.

The nanoemulsions of the non-opioid analgesic formulations provide small-size (less than about 150 nm) oil-in-water emulsion droplets with a surface-area-to-volume ratio that allows for effective loading of multiple payloads and targeted release.

The nanoemulsions of the non-opioid analgesic formulations may include active pharmaceutical ingredients such as non-opioid analgesic active pharmaceutical ingredients including celecoxib, acetaminophen, extromethorphan, cyclobenzaprine, benztropine, baclofen, arbaclofen, ritodrine, tizanidine, flurazepam, chlorpheniramine, doxylamine, diphenhydramine, diltiazem, rimantadine, amantadine, memantine, tacrolimus, resveratrol, curcumin, indomethacine, nimusulide, and gabapentin.

The non-opioid analgesic active pharmaceutical ingredient may be present in the non-opioid analgesic formulation in an amount of about 0.05 mg/mL to about 500 mg/mL.

The nanoemulsions of the non-opioid analgesic formulations may include perfluorocarbon (fluorous phase), hydrocarbon (synthetic or natural, organic phase) and water (aqueous) phases implementing Quality by Design (QbD) approaches. In addition, the nanoemulsions may include surfactants such as pluronics and ethoxylated oils (e.g. Pluronic F68, F127, P123, P105 and Cremophor EL), additives such as cationic lipids and quaternary amines such as octadecylamine, dodecyoctylamine, octylamine, and co-solubilizers such as 2-(2-ethoxyethox)ethanol or transcutol, dimethylsulfoxyde (DMSO), ethanol.

As they are generated from GRAS materials, the non-opioid analgesic formulations are non-toxic in vitro and in vivo.

The following non-limiting examples are illustrative.

### Example 1

Macrophage Dynamics in Acute Neuroinflammation after Neuromuscular Injury

The neural-immune interactions driving the process of Wallerian Degeneration (WD) that follows peripheral nerve injury (PNI) involve infiltrating macrophages at the injury. The acute (early phase) neuroinflammation is due to macrophage recruitment, rapid clearance of myelin debris, and Schwann cell activation. Macrophages can assume a pro-inflammatory (M1) or anti-inflammatory/pro-healing (M2) phenotype. M1 macrophages produce pro-inflammatory cytokines such as TNF-α, IFN-g, or IL-12, while M2 macrophages produce anti-inflammatory cytokines such as IL-10, and TGF-β1. When this transition is impaired, chronic inflammation results, increasing the risk of chronic pain. Normal neuro-immune interactions drive the resolution of inflammation and regeneration. Therefore, we focused on therapeutically targeting macrophages for injury-specific immunomodulation to achieve both pain relief and to support tissue recovery and neuroregeneration.

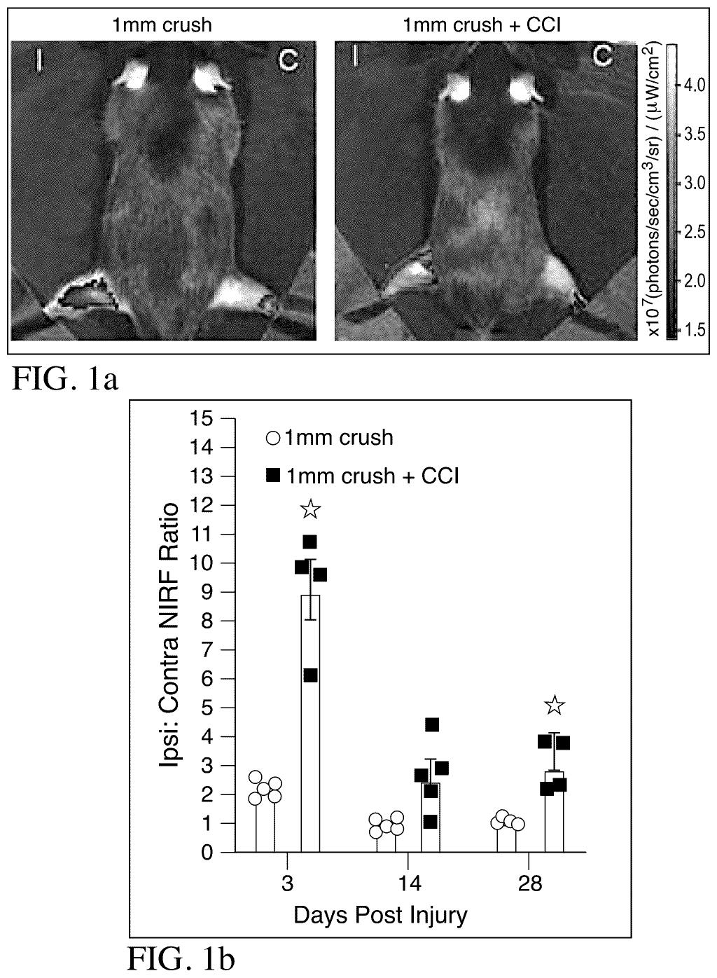

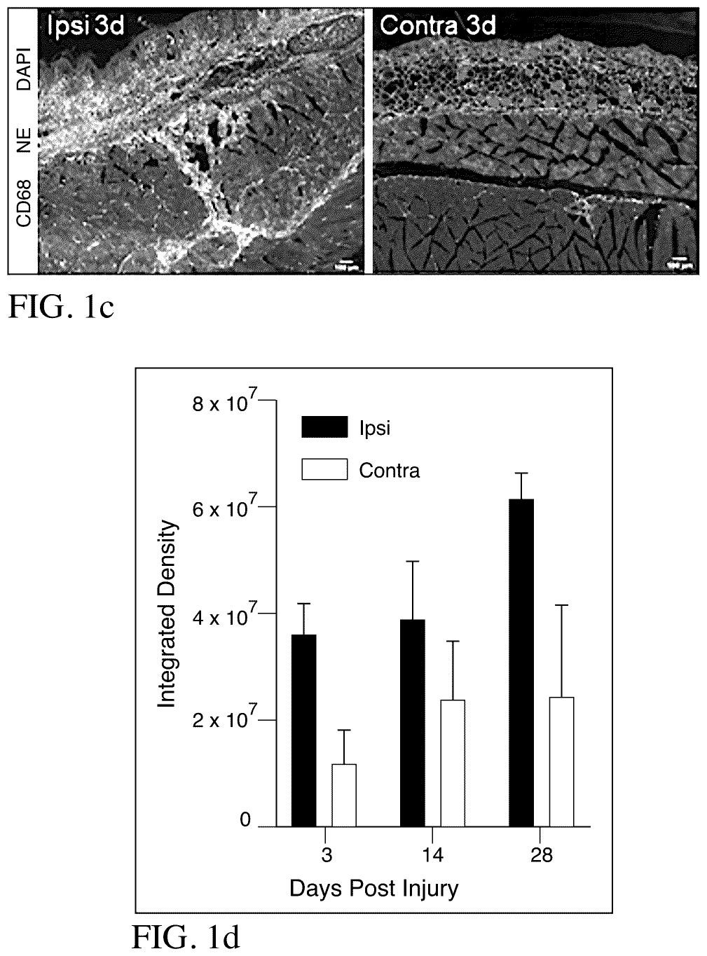

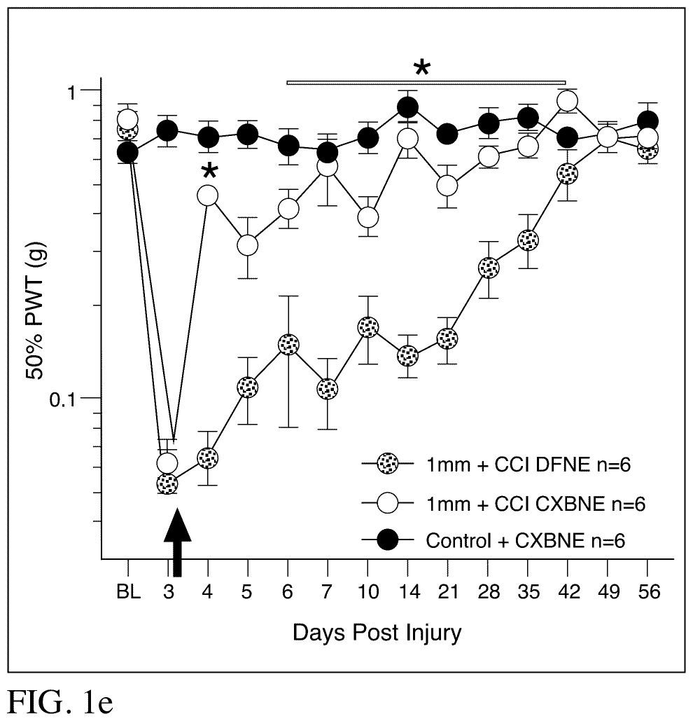

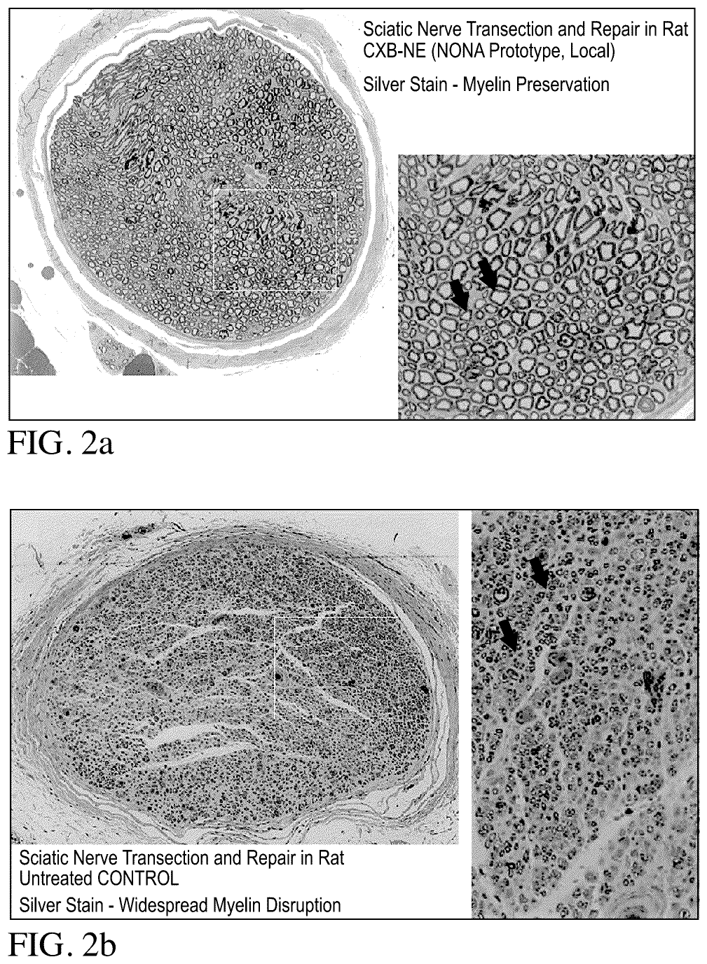

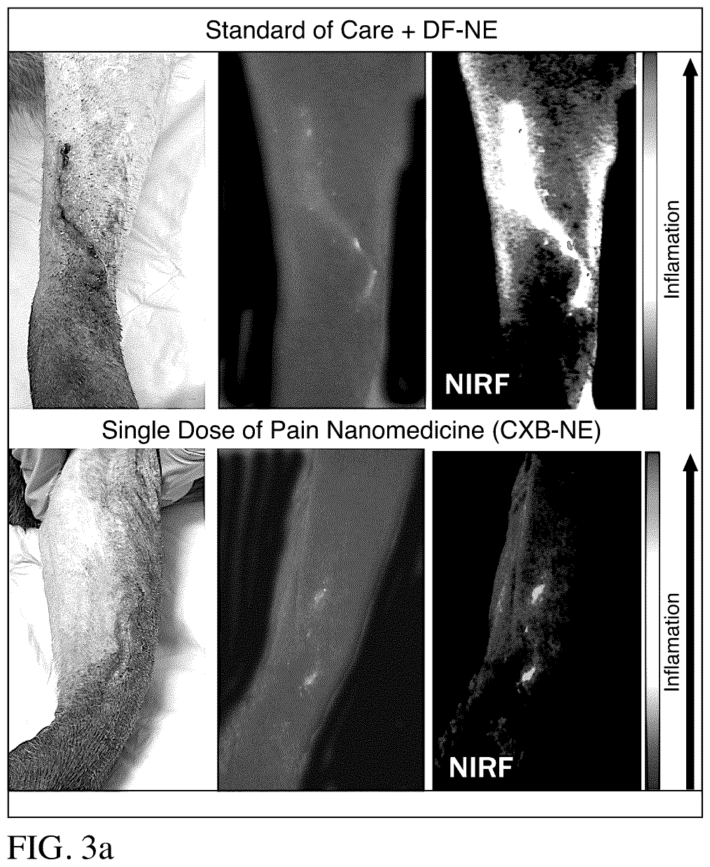

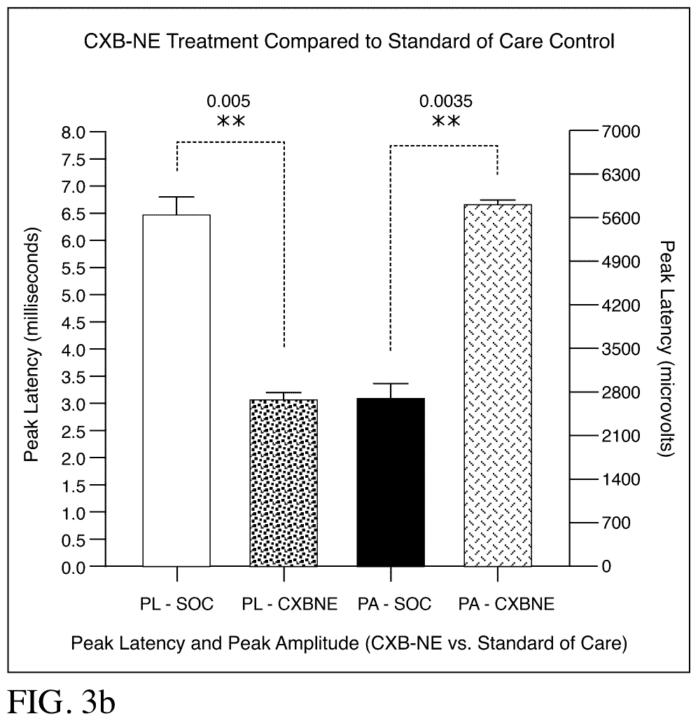

Rodent Models of Neuromuscular Trauma and Nerve Injury: Infiltrating macrophages during acute neuroinflammation actively express cyclooxygenase-2 (COX-2), stimulating the release of prostaglandins (e.g., PgE2) that sensitize neurons. A single low dose of a COX-2 inhibitor (celecoxib) loaded nanoemulsion (CXB-NE, NONA prototype formulation) achieves macrophage-specific COX-2 inhibition resulting in analgesia, reduced neuromuscular inflammation and improved tissue recovery (see FIGS. 1, 2 and 3). Furthermore, CXB-NE produced M1 to M2 macrophage phenotype shift, consequently promoting axonal regeneration marker expression. Chronic constriction injury (CCI) of the rat sciatic nerve allowed us to demonstrate that near-infrared fluorescently labeled nanoemulsion (NIRF-NE) can effectively reveal macrophage infiltration and serve as real-time surrogate biomarkers for neuroinflammation. Pain behavior returning to baseline coincided with decreased NIRF signal in CXB-NE treated animals vs. NIRF labeled drug-free nanoemulsion (DF-NE negative control). This corresponded to reduced macrophage infiltration measured by histological assessment of macrophage infiltration. These findings were validated in NHP (see FIG. 3). Our mouse model of neuro-muscular trauma (developed under an award W81XWH-21-1-0197, PI: Shepherd), confirms macrophages are a key cellular target in neuro-muscular trauma. Macrophage infiltration in contused muscle associated with hypersensitivity severity was revealed by increased NIRF signal (FIG. 1a-b). Macrophages surround injured muscle fibers histologically (FIG. 1c-d). Sustained pain relief (>40 days) was achieved in this model following a single dose of CXB-NE (FIG. 1e). Further, confirmation of the pro-regenerative effects of macrophage-specific COX-2 inhibition was obtained in a rat sciatic nerve transection and repair (SN-TR) model. Locally administered CXB-NE promoted axon survival and myelin preservation following SN-TR injury while untreated controls were associated with extensive myelin disruption and axonal loss (FIG. 2)

### Example 2

### Non-Human Primate (NHP) Radial Nerve Injury

With NHP Cynomolgus macaques, which have similar physiology to humans and provide a clinically relevant model, we have demonstrated the proof-of-concept for the non-opioid analgesic nanoemulsion formulations, or Non-opioid Nanomedicine Analgesic (NONA prototype formulation-CXB-NE), as a single-dose analgesic. RN transection and repair surgery were performed in two NHPs. Wrist-extension behavior, electrophysiology, and NIRF imaging assessments, along with clinical evaluations, were used to assess the response to a single dose of CXB-NE against the standard of care as a control. With video and photographic documentation, each animal was allowed to maximally extend its wrist from a fixed position to reach for food above its head, and the maximum wrist extension angle was measured. Each video assessment was repeated at least 3 times every week for 6 weeks following surgical intervention. The animals also underwent serial clinical exams every week, including NIRF imaging. In NHPs, the NIRF labeled CXB-NE and DF-NE accumulated at the injury site, consistent with our rodent studies. The NIRF signal was quantified as a surrogate measure of neuromuscular inflammation (FIG. 3a). The animal that received a single i.v. dose of CXB-NE at the point of surgery showed marked improvement in behavior, reduced inflammation by NIRF imaging, and accelerated wound healing compared to standard of care (i.m. meloxicam daily) who received drug-free control (DF-NE), FIG. 3a. CXB-NE improved nerve sensory conduction. Elevated Peak Latency (PL) is evidence of persistent neuroinflammation, neuroma, or myelin damage/loss, all of which can interfere with or impair nerve conduction. Increased Peak Amplitude (PA) is associated with axonal sprouting, which is evidence of regenerating axons (FIG. 3b)

TABLE 1

| Product Properties | Minimum Acceptable Result | Ideal Result |
|---|---|---|
| Primary Indication | Relief of battlefield neuromuscular | Relief of general acute pain of mixed |
| | pain andor post-operative pain | nociceptive/peripheral neuropathic origin |
| Patient Population | Adults aged 18 and older with neuro- | Adults, adolescents and children (ages ≥2 |
| | muscular pain and/or post-operative | to 12) with general acute pain of mixed |
| | pain | nociceptive/peripheral neuropathic pain |
| Treatment Duration | Acute | Acute, prevents chronic pain onset |
| Delivery Mode | i.v. | i.v. and s.c. |
| Dosage Form | Nano emulsion, lyophilized powder | Nanoemulsion, lyophilized powder |
| Regimen | 1x/72 hrs | 1x/week+ |
| Efficacy | A 60% decrease in pain score in 50% | A 80% decrease in pain score in 60% of |
| | of patients | patients |
| Risks/Side Effects | Minimal grade 3 or 4 adverse events; | No grade 2, 3 or 4 adverse events and |
| | no cardiovascular or respiratory risks, | well tolerated overall, devoid of opioid |
| | or target organ toxicities, no | and NSAID side effects |
| | central/cognitive effects | |

The following product description is consistent with Example 2.

Product Description: Non-Opioid Nanomedicine Analgesic (NONA) for Prolonged Pain Relief (>72 Hours) with a Single Dose.

The indications for use of a product of this description is the management of mixed battlefield pain of mixed-nociceptive/peripheral neuropathic origin, defined as pain that is self-limited (wanes as a wound or disease state heals) and generally requires treatment for no more than up to a few weeks. Furthermore, the non-opioid analgesic nanoemulsion formulations will be produced sterile, and in two forms: 1) an injectable nanoemulsion, liquid form, if i.v. access is available and 2) a powder form for durability, storage, and easy use that can be reconstituted with saline in the field by a medic or fellow servicemember for treatment of cute trauma pain by a direct application to the wound or s.c. in the absence of available i.v. access. Importantly, the powdered form can also be injected, i.v. upon reconstitution.

### Example 3

### Anti-Inflammatory Efficacy and Macrophage Phenotype Assays In Vitro

COX-2 inhibition and dosing/timing with non-opioid analgesic formulations based on celecoxib-nanoemulsions (CXB-NE) will be evaluated against drug-free control or free drug in vitro. The ability of CXB-NE to alter macrophage pro-inflammatory activity will be evaluated by assaying for specific cytokines and intracellular markers. Phenotype Analysis: The ability of drug-loaded NEs to alter the macrophage phenotype will be evaluated by assessing M1 and M2-specific markers. Macrophages will be exposed to different treatments (drug-loaded and drug-free NEs, free drugs and vehicle controls) for 24 h, followed by LPS/IFN-γ or IL-4/IL-13 priming, based on our previous uptake studies with CXB-NE.

### Example 4

### In Vivo Rodent Studies

The non-opioid analgesic formulations will be evaluated in mouse neuromuscular trauma model and separately rat sciatic nerve transection and repair (SN-TR) models, followed by validation in an established NHP radial nerve transection and repair (RN-TR) injury model. Pain Behavior Testing-Mice: Comprehensive behavioral analysis will be performed using established reflexive and non-reflexive assays to monitor emergence of acute and chronic pain. von Frey testing will be our reflexive measure carried out weekly from baseline to 8 weeks post-injury. Grip strength assessment of hindlimb function will also be carried out weekly. Non-reflexive pain measures will include mechanical conflict-avoidance, and a thermal gradient ring assay.

The following provide some more particular description of the content and significance of the Figures.

In FIGS. 1a-1e, A single dose of CXB-NE produces 40 days analgesia in a mouse neuromuscular trauma model. FIGS. 1 (a-b) NIRF shows increased signal in injured (left) hindlimbs with 1 mm muscle crush+CCI (polytrauma), vs 1 mm muscle crush+sham nerve injury. (c-d) IHC of 1 mm crush+CCI skin/muscle shows DF-NE+ CD68+ macrophage infiltration of fascia surrounding the injured muscle 3d post-injury. (e) Treatment of polytrauma mice with celecoxib (CXBNE) 3d post-injury attenuates von Frey hypersensitivity (PWT: paw withdrawal threshold. (Data source: Dr. Andrew Shepherd, MD Anderson.)

In FIGS. 2a-2b, CXB-NE applied locally (at the site of nerve injury) promotes myelin preservation after SN-TSR in a rodent model. Panel A shows sliver staining of nerve segment 10 mm distal to transection and repair clearly delineating preserved myelin. In comparison, control (Panel b, untreated nerve sections) show extensive myelin disruption and axon loss. (Data Source: Dr. Vijay S. Gorantla, WFIRM.)

In FIGS. 3a and 3b, results show a single dose of CXB-NE immediately following RN transection and repair surgery in NHPs. The control animal received drug-free nanoemulsion (DF-NE) and standard of care. Top: Control clinical image of surgical site in animal receiving standard of care and single dose of drug free NE revealing NIRF inflammation NIRF; Bottom: Clinical image of surgical site in animal treated with CXB-NE and NIRF imaging. Marked reduction in signs of inflammation is observed in CXB-NE treated animal both by NIRF imaging and clinical observation. Panel (2): Sensory Nerve Conduction in Radial Nerve. Peak Latency (PL): CXB-NE treatment was associated with decreased PL after nerve transection and repair, as compared to standard of care (SOC) control (p=0.005**). Peak Amplitude (PA): CXB-NE treatment was associated with increased PA after nerve transection and repair, as compared to standard of care (SOC) (p=0.0035**). Unpaired t test with Welch's correction. (Data Source: Dr. Vijay S. Gorantla, WFIRM.)

I claim:

1. A non-opioid analgesic formulation comprising an active pharmaceutical ingredient and a stabilized nanoemulsion,
wherein the active pharmaceutical ingredient comprises a non-opioid analgesic;
where the nanoemulsion comprises: a hydrocarbon lipid; a perfluorocarbon; water and/or buffered saline; a nonionic surfactant; and optionally a quaternary ammonium compound and/or a lipid; and where droplets of the non-opioid analgesic formulation have a droplet size of from about 90 nm to about 120 nm and wherein the diameter of the droplets does not change by more than 20% upon storage for at least 12 months.

2. The non-opioid analgesic formulation of claim 1, where the formulation is used as a single dose form.

3. The non-opioid analgesic formulation of claim 2, where the single dose form is a parenteral form.

4. The non-opioid analgesic formulation of claim 2, where the single dose form is in liquid form.

5. The non-opioid analgesic formulation of claim 2, where the single dose form is in powdered form.

6. The non-opioid analgesic formulation of claim 1, where the non-opioid analgesic is selected from the group consisting of celecoxib, acetaminophen, extromethorphan, cyclobenzaprine, benztropine, baclofen, arbaclofen, ritodrine, tizanidine, flurazepam, chlorpheniramine, doxylamine, diphenhydramine, diltiazem, rimantadine, amantadine, memantine, tacrolimus, resveratrol, curcumin, indomethacine, and nimusulide.

7. A method of preparing the non-opioid analgesic formulation of claim 1 comprising
(a) pre-mixing a solution including a hydrocarbon, and a co-solubilizer to form a pre-mix;
(b) adding a perfluorocarbon and a surfactant aqueous solution to the pre-mix to form a pre-emulsion solution;
(c) mixing and blending of the pre-emulsion solution to form a crude emulsion;
(d) adding a surfactant aqueous solution to the crude emulsion;
(e) incorporating the active pharmaceutical ingredient into a blend of surfactants, oils and co-solubilizers by low energy mixing/pre-emulsification; and
(f) emulsifying the crude emulsion via multiple passages through a microfluidizer.

8. A method of preparing the non-opioid analgesic formulation of claim 1 comprising lyophilizing the nanoemulsion into sterile powder without the addition of cryoprotectants.

9. A method of preparing the non-opioid analgesic formulation of claim 1 comprising scaling up the analgesic formulation from about 25 mL/batch to about 500 mL/batch.

10. A method of preparing the non-opioid analgesic formulation of claim 1 comprising incorporating the formulation into hydrogels for local delivery.

11. A method of preparing the non-opioid analgesic formulation of claim 1 comprising incorporating fluorescent dyes and APIs into the same non-opioid analgesic formulation.

12. A method of preparing the non-opioid analgesic formulation of claim 1 comprising incorporating multiple APIs into the same non-opioid analgesic formulation.

13. A method of preparing the non-opioid analgesic formulation of claim 1 comprising reconstituting the lyophilized product back into a liquid form for parenteral and local administration.

14. A method of using the non-opioid analgesic formulation of claim 1 comprising administering a single parenteral dose of the formulation to a patient suffering trauma pain in battlefield.

15. The method of using the non-opioid analgesic formulation of claim 1 comprising administering a single parenteral dose of the formulation to a patient in battlefield, where effective trauma pain relief lasts at least 72 hours following administration of the single parenteral dose.

16. The method of using the non-opioid analgesic formulation of claim 1 comprising administering a single parenteral dose of the formulation to a patient in battlefield, where the patient does not suffer systemic and performance-limiting side effects or cardiovascular and respiratory liabilities following administration of the single parenteral dose.

17. The method of using the non-opioid analgesic formulation of claim 1 comprising administering a single local dose of the formulation to a patient in battlefield, where the patient does not suffer systemic and performance-limiting side effects or cardiovascular and respiratory liabilities following administration of the single parenteral dose.

18. A method of local immunomodulation by administering the non-opioid analgesic formulation of claim 1.
